(19) United States
(12) Patent Application Publication
(10) Pub. No.: US 2026/0027143 A1
(43) Pub. Date: Jan. 29, 2026

(54) FERMENTATION COMPOSITION WITH NMN ANTI-AGING EFFECT

(71) Applicant: BIOZYME BIOTECHNOLOGY CORP., Banqiao City (TW)

(72) Inventors: Cheng HUANG, Banqiao City (TW); Yi-chung LAI, Banqiao City (TW); Po-ju LAI, Banqiao City (TW)

(21) Appl. No.: 18/997,058

(22) PCT Filed: Aug. 3, 2022
(86) PCT No.: PCT/CN2022/110018

§ 371 Date: Jan. 18, 2025

(51) Int. Cl. A61K 31/706; A61K 35/745; A61K 35/747; A61K 36/062; A61K 36/064; A61K 36/48; A61K 36/54; A61K 36/81; A61P 39/00

(52) U.S. Cl. CPC A61K 31/706; A61K 35/745; A61K 35/747; A61K 36/062; A61K 36/064; A61K 36/48; A61K 36/54; A61K 36/81; A61P 39/00; A61K 2236/15; A61K 2236/19; A61K 2236/39; A61K 2236/51; A61K 2236/53

(57) ABSTRACT

A fermentation composition with an NMN anti-aging effect, and the use thereof in the preparation of an anti-aging drug. The fermentation composition is prepared by subjecting vegetable and fruit raw materials to a special fermentation preparation method (comprising the steps of extracting vegetables and fruit, breaking walls under negative pressure, performing fermentation at a low temperature, performing deep fermentation, performing chelation fermentation, drying, granulating, etc.), wherein the vegetable and fruit raw materials comprise truffles, mushroom fruiting bodies, avocados, tomato

### CROSS-REFERENCE TO RELATED APPLICATIONS

This application is a U.S. National Stage of International Application No. PCT/CN2022/110018, filed on Aug. 3, 2022, for which priority is claimed under 35 U.S.C. § 120; the entire contents of all of which are hereby incorporated by reference.

### FIELD OF THE INVENTION

The present invention provides a fermentation composition, which can effectively delay the expression of aging-related genes, improve the content of sod and gsh of antioxidant indexes, reduce the oxide concentration of tissues and organs, and achieve the anti-aging effect.

### BACKGROUND OF THE INVENTION

NMN (nicotinamide mononucleotide) is one of the derivatives of vitamin B3 (nicotinic acid), also an intermediate product of NAD⁺biosynthesis, which is a biologically active nucleotide formed by the reaction of a phosphate group and a nucleoside containing ribose and nicotinamide.

At present, NMN has been approved by Japan for food additives, Japan is a high-concentration NMN substance in a purification manner after NMM is produced by fermentation technology, but has no other national approval of NMN for food, so that NMN currently required by health-care products is NMN precursor added to vitamin B3, while vitamin B3 is NMN precursor, but still requires in-vivo enzymes for conversion before functioning to generate NAD⁺to have activity.

However, the health-care product required by commercially available NMN is actually added with vitamin B3, and in the aged population, the metabolic rate in the vitamin B3 cannot be completely converted into NAD+, so it is difficult to take vitamin B3 to delay aging as NMN. In addition, in some physician studies, NMN can mainly improve the regeneration capacity of cells, but has no complete research on whether the cancer cells have the same effect, and therefore, there may be eating risks.

In view of this, there is an urgent need in the relevant art to develop a composition that is similar to the NMN component and has the potential to delay aging.

### SUMMARY OF THE INVENTION

In order to solve the above problems, the present invention provides a method for preparing a fermentation composition having an NMN delayed aging effect, comprising:

(1) a vegetable and fruit extraction step: respectively carrying out squeezing treatment on various vegetables and fruits in a physical manner, pressing vegetables and fruits until the fruits or the fruiting bodies are in a fragment shape, and meanwhile, reserving juice and pomace or fruiting bodies to obtain a plurality of first extracts;

(2) a negative pressure wall breaking method: respectively adding 0.15-1.5% of brown sugar to enhance the isotonicity of the first extract, and simultaneously utilize a negative pressure extraction method in a near-vacuum environment of 35-45 cmHg, continuously extracting for 3-15 days, this process will disrupt the cell walls of fruits and vegetables, releasing intracellular nutrients and polysaccharides to obtain multiple secondary extracts;

(3) a low-temperature fermentation stage: a yeast strain, at a ratio of 0.5-1.5% (10⁷ CFU/mL), is inoculated into the secondary extracts prepared in step (2), the fermentation is carried out at a controlled temperature of 2-12° C. for 3-15 day; during this stage, the yeast remains in a low-activity state, allowing enzymatic activity to dominate over microbial activity, which slows down carbohydrate consumption, this process aids in preservation during fermentation, enhances the decomposition of fibrous polysaccharides, and promotes the breakdown of cell walls, ultimately yielding multiple primary fermentation products;

(4) a deep fermentation stage: a lactic acid bacterium, at a ratio of 0.5-1.5% (10⁷ CFU/mL), along with 0.15-0.25% isomalto-oligosaccharides, is inoculated into the primary fermentation products prepared in step (3); the fermentation is conducted at a controlled temperature of 20-35° C. for 5-25 days, during this stage, the lactic acid bacteria metabolize sugars to produce lactic acid, which lowers the pH to achieve a preservative effect, simultaneously, the increased acidity reduces viscosity, resulting in the production of multiple secondary fermentation products;

(5) a chelation fermentation stage: an acetic acid bacterium, at a ratio of 0.5-1.5% (10⁷ CFU/mL), along with 2.5-5.5% D-sorbitol, is inoculated into the secondary fermentation products prepared in step (4); the fermentation process is carried out at a controlled temperature of 10-25° C. for 5-20 days, during this stage, in an oxygen-rich environment, glucose is consumed to produce acetic acid, ethanol is metabolized, acidity is increased, and viscosity is reduced, resulting in the production of multiple tertiary fermentation products; and

(6) drying and granulating: the tertiary fermentation products prepared in step (5) are individually filtered to obtain multiple fermentation liquids, which are retained, these fermentation liquids are then mixed in equal proportions and subjected to spray drying to form granules, resulting in the fermentation composition.

The present invention further provides a fermentation composition having an NMN postponed aging effect, comprising a vegetable and fruit raw material, wherein the vegetable and fruit raw material comprises one or more of truffle, mushroom fruiting body, avocado, tomato or edamame, and the fermentation composition is obtained by a special fermentation preparation method.

In an embodiment of the present invention, the preparation method of the fermentation composition with the NMN delayed aging effect further comprises the step of mixing the vegetable and fruit with water.

In an embodiment of the present disclosure, the preparation method of the fermentation composition with the NMN delayed aging effect, wherein the vegetable and fruit comprise one or more of truffle, mushroom fruiting body, avocado, tomato or edamame.

In the embodiment of the present invention, the preparation method of the fermentation composition with the NMN delayed aging effect, wherein when the vegetables and fruits in the vegetable and fruit extraction step are truffle or mushroom fruiting bodies, the vegetable and fruit extraction step further comprises a step of soaking in hot water at 65-100° C. for 25-35 minutes before pressing in a physical manner.

In the embodiment of the present invention, the preparation method of the fermentation composition with the NMN delayed aging effect comprises the following steps: mixing truffle or mushroom fruiting bodies with water in a ratio of 1:40; the ratio of the avocado to the water is 1:25; the ratio of the tomatoes to the water is 1:1; and the ratio of the edamame to the water is 1:30.

In an embodiment of the present invention, the preparation method of the fermentation composition with the NMN delayed aging effect comprises the following steps: the pH value of the secondary extract is 5.5-7; the pH value of the first fermentation product is 5-6.5; the pH value of the second fermentation product is 4.5-5.5; and the pH value of the tertiary fermentation product is 3-3.5.

In an embodiment of the present invention, the preparation method of the fermentation composition with the NMN delayed aging effect comprises the following steps: the secondary extract has a sugar degree of 21-28° BX; the sugar degree of the first fermentation is 15-34° BX; the sugar degree of the second fermentation is 15-38° BX; and the sugar degree of the tertiary fermentation is 10-50° BX.

In an embodiment of the present invention, a method for preparing a fermentation composition having an NMN delayed aging effect, wherein the lactic acid bacteria are selected from one of Lactobacillus plantarum (L. plantarum), Lactobacillus bulgaricus (L delbrueckii), Lactococcus lactis (L Lactis), Lactobacillus acidophilus or B. bifidum.

In an embodiment of the present invention, a method for preparing a fermentation composition having an NMN postponed aging effect, wherein the saccharomyceses or the acetic acid bacteria are one selected from the group consisting of S fibuligera, S cerevisited, P facens, S. pombe, A hansenii, A xylimum, or A suboxyDans.

In the embodiment of the present invention, the fermentation composition with NMN postponed aging effect is used for preparing a drug for postponing aging, delayed aging refers to postponing the expression of aging-related genes, improving the content of SOD and GSH of antioxidant indexes, and reducing the oxide concentration of tissues and organs.

It is a primary object of the present invention to provide a use of a fermentation composition with NMN postponed aging for the preparation of a delayed aged drug, wherein the drug can be used to postpone aging of the heart, brain, liver.

### BRIEF DESCRIPTION OF THE DRAWINGS

FIGS. 1A-1D show the difference in sugar degree, pH value, cellulose and polysaccharide body of the temperature gradient fermentation technology and the traditional fermentation technology.

FIGS. 2A-2C show the results of the expression quantity of the fermentation composition with NMN postponed aging for mouse hematocellular gene expression of the present invention.

FIGS. 3A-3B are a result diagram of a fermentation composition with NMN delayed aging effect on blood antioxidant index concentration of mice according to the present invention.

FIGS. 4A-4C are graphs of oxide concentrations in mouse tissue organs for fermentation compositions with NMN postponed aging efficacy of the present invention.

FIG. 5 is a result diagram of the fermented composition with NMN delayed aging effect of the present invention for prolonging the life test of mice.

### DETAILED DESCRIPTION OF THE INVENTION

The present disclosure will be fully understood by the following examples, so that those skilled in the art may complete the implementation of the present disclosure, however, the implementation of the present disclosure may not be limited by the following embodiments, and those skilled in the art may still derive other embodiments according to the spirit of the embodiments disclosed herein, and the embodiments are all within the scope of the present disclosure.

### Example 1: Preparation Method of Fermentation Composition Similar to NMN Activity

Using lactic acid bacteria, yeast, and acetic acid bacteria, temperature gradient fermentation regulation and control was conducted with various types of fruits and vegetables, including truffles, avocados, tomatoes, and edamame. After fermentation, the resulting fermentation broth was filtered, retaining only the filtrate. The temperature-gradient fermentation control process for the fermentation composition includes the following steps:

(1) a vegetable and fruit extraction step: respectively carrying out squeezing treatment on various vegetables and fruits in a physical manner, pressing vegetables and fruits until the fruits or the fruiting bodies are in a fragment shape, and meanwhile, reserving juice and pomace or fruiting bodies to obtain a plurality of first extracts;

(2) a negative pressure wall breaking method: respectively adding 0.15-1.5% of brown sugar to enhance the isotonicity of the first extract, and simultaneously utilize a negative pressure extraction method in a near-vacuum environment of 35-45 cmHg, continuously extracting for 3-15 days, this process will disrupt the cell walls of fruits and vegetables, releasing intracellular nutrients and polysaccharides to obtain multiple secondary extracts;

(3) a low-temperature fermentation stage: a yeast strain, at a ratio of 0.5-1.5% (10⁷ CFU/mL), is inoculated into the secondary extracts prepared in step (2), the fermentation is carried out at a controlled temperature of 2-12° C. for 3-15 day; during this stage, the yeast remains in a low-activity state, allowing enzymatic activity to dominate over microbial activity, which slows down carbohydrate consumption, this process aids in preservation during fermentation, enhances the decomposition of fibrous polysaccharides, and promotes the breakdown of cell walls, ultimately yielding multiple primary fermentation products, wherein the yeast strain is selected from one of S. fibuligera, Saccharomyces cerevisiae (S. cerevisiae), Pichia pastoris (P. Facens) or Poppy yeast (S. Pombe);

(4) a deep fermentation stage: a lactic acid bacterium, at a ratio of 0.5-1.5% (10⁷ CFU/mL), along with 0.15-0.25% isomalto-oligosaccharides, is inoculated into the primary fermentation products prepared in step (3); the fermentation is conducted at a controlled temperature of 20-35° C. for 5-25 days, during this stage, the lactic acid bacteria metabolize sugars to produce lactic acid, which lowers the pH to achieve a preservative effect, simultaneously, the increased acidity reduces viscosity, resulting in the production of multiple secondary fermentation products, wherein the lactic acid bacteria are selected from one of the following species: Lactobacillus plantarum (L. plantarum), Lactobacillus delbrueckii (L. delbrueckii), Lactococcus lactis (L. lactis), Lactobacillus acidophilus (L. acidophilus), or Bifidobacterium bifidum (B. bifidum);

(5) chelation fermentation stage: in an acetic acid bacteria, 0.5-1.5% (10⁷ CFU/mL); 2.5-5.5% D-sorbitol are respectively implanted into a plurality of second fermentation products, the fermentation temperature is controlled at 10-25° C. the fermentation is continued for 5-20 days, the oxygen consumption environment consumes glucose to generate acetic acid in this stage, ethanol is consumed, the acidity is increased, the viscosity is reduced, and a plurality of tertiary fermentation substances are obtained, wherein the acetic acid bacteria are selected from one of the following species: Acetobacter hansenii (A. hansenii), Acetobacter xylimum (A. xylim), or Acetobacter suboxydans (A. suboxydans); and

(6) drying and granulating: filtering the tertiary fermentation product prepared by chelating fermentation in step (5) to obtain a plurality of fermentation broth, retaining the fermentation broth, mixing the fermentation broth at equal proportions, and then spray drying and granulating to obtain the fermentation composition.

In the first embodiment, the yeast used is Saccharomycopsis fibuligera (S. fibuligera) as an example. Other yeasts, such as Saccharomyces cerevisiae (S. cerevisiae), Pichia faciens (P. faciens), or Schizosaccharomyces pombe (S. pombe), are also applicable to this invention. For lactic acid bacteria, Lactobacillus plantarum (L. plantarum) is provided as an example. Other lactic acid bacteria, such as Lactobacillus delbrueckii (L. delbrueckii), Lactococcus lactis (L. lactis), Lactobacillus acidophilus (L. acidophilus), or Bifidobacterium bifidum (B. bifidum), are equally suitable for this invention. Regarding acetic acid bacteria, Acetobacter hansenii (A. hansenii) is used as an example. Other acetic acid bacteria, such as Acetobacter xylimum (A. xylimum) or Acetobacter suboxydans (A. suboxydans), are also applicable to this invention.

In one embodiment, the vegetable and fruit extraction step further comprise the step of mixing the vegetable and fruit with water.

In one embodiment, the vegetable and fruit comprise truffle, mushroom fruiting body, avocado, tomato or edamame.

In an embodiment, when the vegetables and fruits in the vegetable and fruit extraction step are truffle or mushroom fruiting bodies, before squeezing in a physical manner, the vegetable and fruit extraction step further includes a step of soaking in hot water at 65-100° C. for 25-35 minutes.

In an embodiment, the mixing mass ratio of the vegetables and fruits to the water is respectively as follows: the ratio of the truffle or the mushroom fruiting bodies to the water is 1:40; the ratio of the avocado to the water is 1:25; the ratio of the tomatoes to the water is 1:1; and the ratio of the edamame to the water is 1:30.

In an embodiment, the pH value of the secondary extract is 5.5-7; the pH value of the first fermentation product is 5-6.5; the pH value of the second fermentation product is 4.5-5.5; and the pH value of the tertiary fermentation product is 3-3.5.

In one embodiment, the secondary extract has a sugar degree of 21-28° BX; the first fermentation product has a sugar degree of 15-34° BX; the second fermentation product has a sugar degree of 15-38° BX; and the tertiary fermentation product has a sugar degree of 10-50° BX.

### Embodiment 2. Temperature Gradient Fermentation Regulation and Control Experiments

The temperature gradient fermentation regulation technique of the present invention differs significantly from traditional fermentation methods. This innovative temperature gradient fermentation technique provides a more complete fermentation effect, while preserving a greater amount of cellulose and releasing more intracellular polysaccharides.

In comparison to traditional fermentation methods, the experimental results for the temperature gradient fermentation technique (T) and the traditional fermentation group (C) are as follows: the traditional fermentation temperature is maintained between 25-30° C.

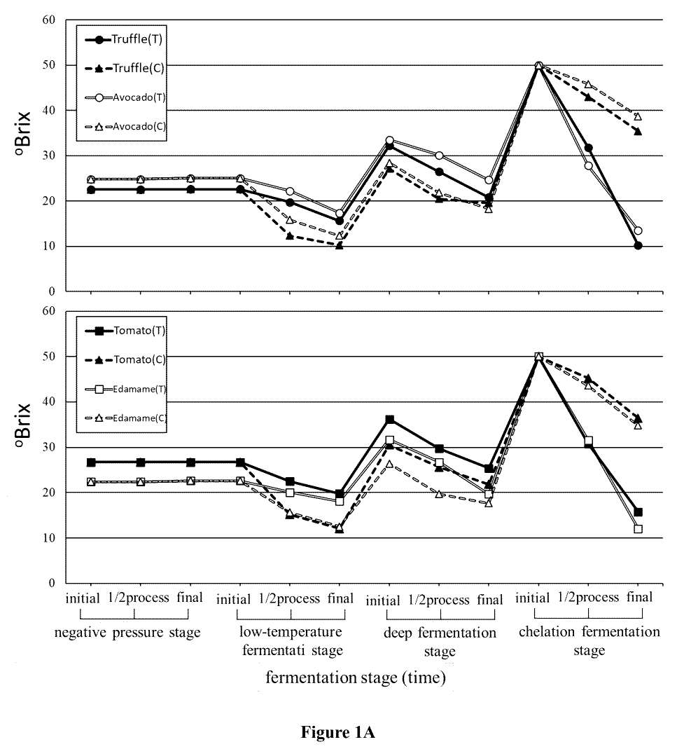

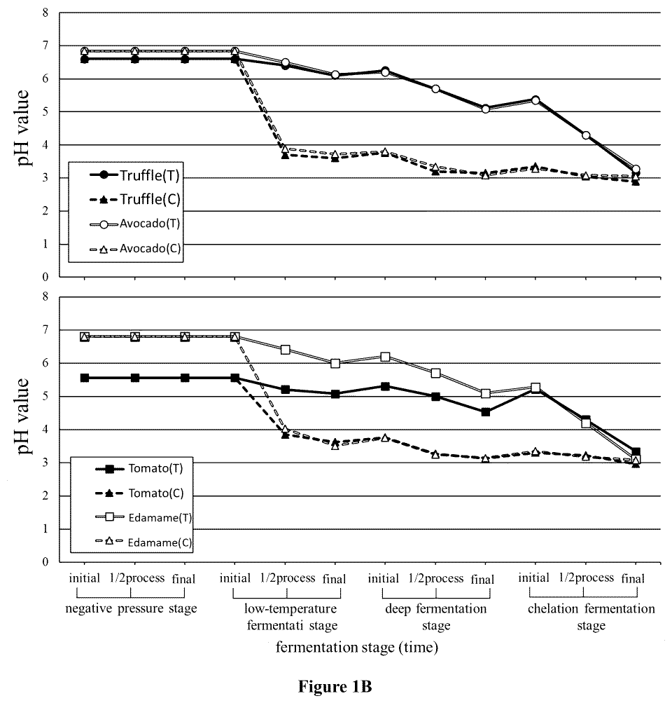

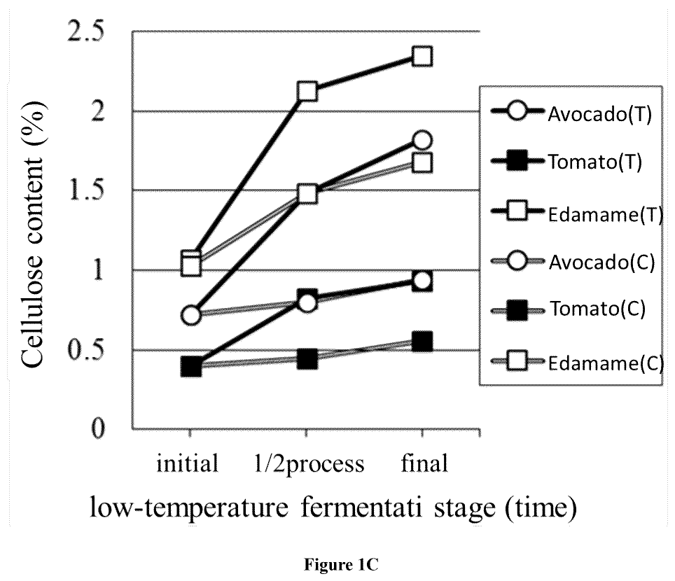

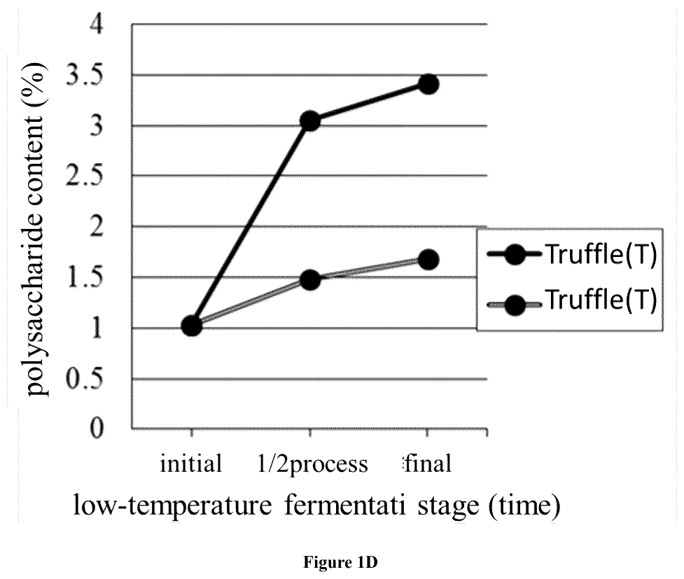

Temperature Gradient Fermentation Regulation and Control
By regulating the temperature during the fermentation process, the activity of microbial strains
and enzymes is modulated, influencing the rate of sugar consumption. The resulting product is
characterized by reduced sugar content and anti-aging properties.

| | (1) negative pressure | (2) low-temperature | (3) deep | (4) chelation |
|---|---|---|---|---|
| Stage | wall breaking method | fermentation stage | fermentation stage | fermentation stage |
| Strain | no | S. fibuligera | L. plantarum | A. hansenii |
| Proportion | no | 0.75~1%(10⁷ CFU/ml) | 0.75~1%(10⁷ CFU/ml) | 0.75~1%(10⁷ CFU/ml) |
| Sugar | brown sugar | no | Isomalto-oligosaccharides | D-Sorbitol |
| Sugar ratio | 0.5% | no | 0.2% | 3%~5% |
| Composition | truffle fruiting bodies:water (1:40) | | | |
| ratio | avocado:water (1:25) | | | |
| | tomato:water (1:1) | | | |
| | edamame:water (1:30) | | | |
| | (The anti-aging research findings pertain to an equally | | | |
| | proportioned mixture of four different fermentation liquids) | | | |
| Brix | truffle 22~24 | truffle 22~34→15~17 | truffle 30~34→15~25 | truffle 50→10~15 |
| (°Bx) | avocado 23~25 | avocado 23~25→16~19 | avocado 32~35→17~27 | avocado 50→12~16 |
| | tomato 26~28 | tomato 26~28→19~22 | tomato 35~38→20~30 | tomato 50→14~18 |
| | edamame 21~23 | edamame 21~23→16~19 | edamame 30~33→15~25 | edamame 50→10~13 |
| Ph value | truffle 6.5~7 | truffle 6.5~7→6~6.5 | truffle 6~6.5→5~5.5 | truffle 5~5.5→3~3.5 |
| | avocado 6.5~7 | avocado 6.5~7→6~6.5 | avocado 6~6.5→5~5.5 | avocado5~5.5→3~3.5 |
| | tomato 5.5~6 | tomato 5.5~6→5~5.5 | tomato 5~5.5→4.5~5 | tomato 4.5~5→3~3.5 |
| | edamame 6.5~7 | edamame 6.5~7→6~6.5 | edamame 6~6.5→5~5.5 | edamame 5~5.5→3~3.5 |
| Pressure | 40 cmHg | no | no | no |
| Temperature | Hot water | fermentation | fermentation | fermentation |
| | 70° C. | environment | environment | environment |
| | | 4~10° C. | 25~30° C. | 15~20° C. |
| Days | truffle: 10 days | truffle: 10 days | truffle: 14 days | truffle: 14 days |
| | avocado: 6 days | avocado: 8 days | avocado: 10 days | avocado: 10 days |
| | tomato: 8 days | tomato: 8 days | tomato: 10 days | tomato: 10 days |
| | edamame: 10 days | edamame: 10 days | edamame: 14 days | edamame: 14 days |
| Purpose | Increase osmotic | Maintain the | Increase the | In an |
| | pressure to enhance | microorganism in a | temperature to the | oxygen-deprived |
| | the release of | low-activity state, | optimal level for the | environment, |
| | intracellular | allowing enzyme | microorganism to | glucose is |
| | substances. | activity to outweigh | enhance its activity. | consumed to |
| | Utilize vacuum | microbial activity. | The lactic acid bacteria | produce acetic acid, |
| | methods to | Slow down the | consume sugars to | and ethanol is |
| | facilitate | consumption of | produce lactic acid, | consumed (FIG. 1A). |
| | cell wall | sugars and prevent | lowering the pH to | Increase acidity and |
| | rupture. | spoilage during the | exert preservative | reduce viscosity. |
| | | fermentation | effects (FIG. 1B).○ | |
| | | process (FIG. 1A)○ | Increase acidity and | |
| | | Enhance the | reduce viscosity. | |
| | | degradation of fiber | | |
| | | polysaccharides and | | |
| | | promote the | | |
| | | breakdown of cell | | |
| | | walls (FIG. 1C). | | |
| | | Enhance the release | | |
| | | of intracellular | | |
| | | polysaccharides | | |
| | | from fungi (truffles) | | |
| | | (FIG. 1D). | | |
| Directions | Traditional | Traditional | Utilizing temperature | Traditional |
| | extraction methods | extraction methods | gradient fermentation | fermentation |
| | typically use | regulate | control has the effect of | temperatures |
| | physical pressing | fermentation | slowing down carbon | typically range |
| | to break down the | temperature based | source consumption | from 25° C. to 30° C., |
| | cell walls. | on the optimal | during the deep | where the strain |
| | While physical | conditions for the | fermentation phase of | activity is high. |
| | methods can | specific strain, | lactic acid bacteria. By | However, in |
| | provide initial | where the strain | reducing the rate of | temperature |
| | cell wall disruption, | activity typically | carbon source | gradient |
| | negative pressure | surpasses the | consumption, the pH | fermentation, using |
| | extraction is more | enzyme activity. | value decreases more | a range of 15° C. to |
| | effective in | In contrast, | gradually. In traditional | 20° C. allows for a |
| | achieving a more | temperature gradient | fermentation methods, | faster reduction in |
| | complete extraction. | fermentation | the faster consumption | the carbon sources |
| | When combined with | control is designed | of carbon sources leads | of the fermentation |
| | osmotic pressure | to ensure enzyme | to vigorous strain | broth, helping to |
| | from sugar content, | activity exceeds | growth, causing the pH | achieve the desired |
| | this method enhances | that of the strain | to drop quickly to | reduction in product |
| | the overall | during | critical levels, at which | sweetness and |
| | extraction | fermentation. When | point fermentation | addressing the issue |
| | efficiency. | the strain is in a | growth halts. In | of higher sugar |
| | | state of low activity, | contrast, temperature | content typically |
| | | carbon source | gradient fermentation | found in |
| | | consumption is | allows the pH value | conventional |
| | | reduced, providing | during the deep | fermentation |
| | | an auxiliary | fermentation stage to | products. |
| | | preservative effect | be maintained above | Additionally, in an |
| | | during | 4.5 | oxygen-consuming |
| | | fermentation. | | environment, |
| | | Enhanced enzyme | | ethanol can be |
| | | activity, on the | | metabolized to |
| | | other hand, | | generate organic |
| | | accelerates the | | acids, which lowers |
| | | breakdown of cell | | the pH and provides |
| | | walls and chitin, | | a preservative |
| | | leading to a more | | effect, thus |
| | | efficient extraction | | extending the |
| | | of intracellular | | product's shelf life. |
| | | polysaccharides. | | |

The results of the fermentation sugar content are shown in FIG. 1A. In the traditional fermentation group, the sugar content fluctuated significantly, with a rapid decrease at each stage. This quick reduction in sugar levels often led to an early termination of fermentation and failed to inhibit spoilage microorganisms, potentially causing spoilage. In contrast, the temperature gradient fermentation method maintained the sugar content at a level above 15° Bx (Degrees Brix) until the chelation fermentation stage (acetic acid bacteria), at which point the carbon sources were completely fermented, reducing the sugar content to below 15° Bx.

The pH value of the fermentation liquid is shown in FIG. 1B. In the traditional fermentation group, the temperature was optimal for yeast strain growth, leading to rapid strain proliferation and carbon source consumption. As a result, the pH dropped quickly to below 3.8, causing the fermentation process to reach its endpoint too quickly. In subsequent stages, the effect of added strains could not be effectively achieved. Traditionally, pH adjustment would be made before transitioning to the next fermentation stage, using compounds such as calcium carbonate or sodium hydroxide. However, this could unintentionally increase the intake of inorganic mineral salts (e.g., sodium) in the final product. By using the temperature gradient fermentation technique, the yeast fermentation phase is conducted at low temperatures, ensuring the fermentation liquid provides a suitable environment for the growth of other strains before entering subsequent stages. This approach helps extend the fermentation process. During the chelation fermentation stage, the temperature is maintained at 15-20° C., allowing acetic acid bacteria to generate a significant amount of organic acid in an oxygen-limited environment, lowering the pH to around 3 and providing preservative effects.

The results for cellulose content in the fermentation are shown in FIG. 1C. The primary component of the cell walls in herbaceous plants is fiber polysaccharides. In traditional fermentation methods, the activity of the strains exceeds that of the enzymes, causing a rapid decrease in pH and carbon sources, leading to an early endpoint of fermentation. As a result, the majority of the herbaceous plant cell walls are not fully decomposed, and the release of their components is limited. In the temperature gradient fermentation technique, the low-temperature fermentation phase is controlled to ensure that enzyme activity exceeds strain activity, which slows the reduction of carbon sources. This allows for more complete degradation of the cell walls. The results indicate that the cellulose content in the temperature gradient fermentation is higher than in the traditional fermentation technique, suggesting that a higher proportion of the cell walls were decomposed.

The results for polysaccharide content in fermentation are shown in FIG. 1D. The primary component of fungal cell walls is chitin, which must be broken down to release intracellular polysaccharides. In traditional fermentation methods, the activity of the strains exceeds that of the enzymes, leading to a rapid decrease in pH and carbon sources, causing an early fermentation endpoint. As a result, most fungal cells cannot be fully decomposed, limiting the release of their components. In the temperature gradient fermentation technique, the low-temperature fermentation phase is controlled to ensure that enzyme activity exceeds strain activity, which slows the reduction of carbon sources and allows for more complete degradation of the cell wall. The results show that the polysaccharide content in the temperature gradient fermentation is higher than in traditional fermentation, indicating that the proportion of released intracellular polysaccharides is effectively increased.

In FIGS. 1A-1D, (T) is a temperature gradient fermentation technology group; (C) is a conventional fermentation group (the traditional fermentation temperature is controlled at 25-30° C.).

### Example 3. Experimental Animals and Experimental Methods

The NMN-like fermented composition of the present invention demonstrates an effect in delaying aging. According to the “Health Food Aging Delay Health Effect Evaluation Method,” the NMN-like fermented composition of the present invention has shown a positive response in at least one key indicator. This composition can prevent, delay, or inhibit the aging of an individual's appearance, functional decline, and behavioral slowdown. The following animal experiments are based on an adult weighing 60 kg, with dosage conversion calculated using the recommended human intake of 1 g/day per kg of body weight, and the animals were divided into the following 5 groups:
(1) Healthy Group (WT, Wild Type): No sample was added
(2) Aging Group (C, Control, Control Group): No sample was added
(3) Low Dose 0.5× Agent Group (LD, Low Dose)
(4) Dose 1 Dose Group (MD, Medium Dose)
(5.) A high dose of 1.5 times the dose of HD (High Dose)

The mice of the C57BL 6 variety used in animal experiments according to the present invention are 6-12 weeks old, and according to (1) healthy groups, (2) aged groups, (3) low dose groups, (4) medium dose groups, (5) high dose groups, 10 female mice per group and 10 male rats.

The mice were established with D-galactose induced oxidative pressure aging animal models, mice were induced to (1) healthy groups, (2) aged groups, (3) low dose groups, (4) medium dose groups, (5) high dose groups, 10 female mice and 10 male rats per group, and 0.1-0.3 g/kg BW physiological saline water was injected into healthy group mice; injecting 0.1-0.3 g/kg BW of D-galactose solution into the control group, the low-dose group, the medium-dose group, and the high-dose group, inducing 6-10 weeks, and then taking blood to detect the lipid-oxidized malondialdehyde (Malondialdehyde, MDA).

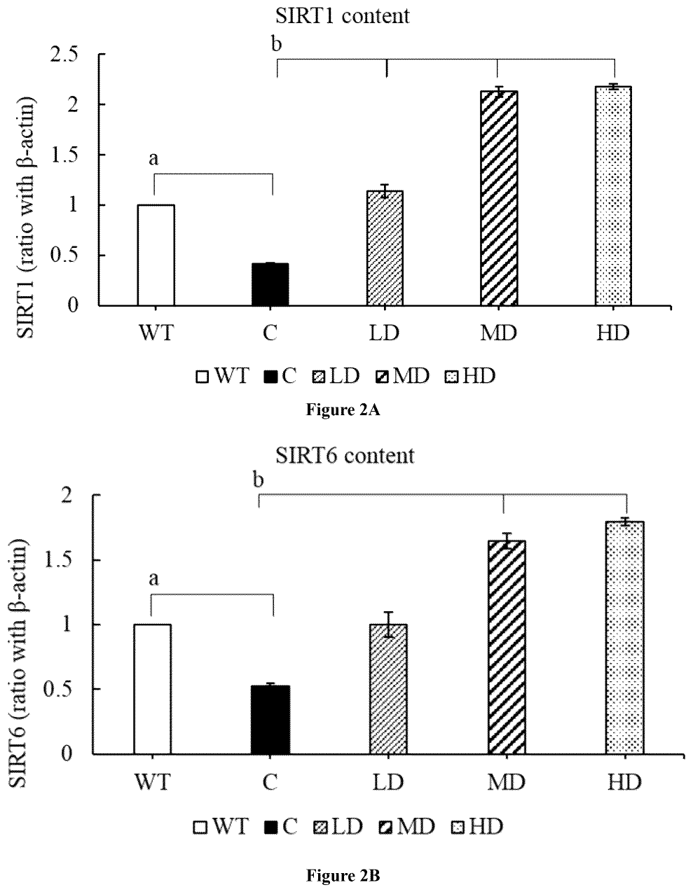

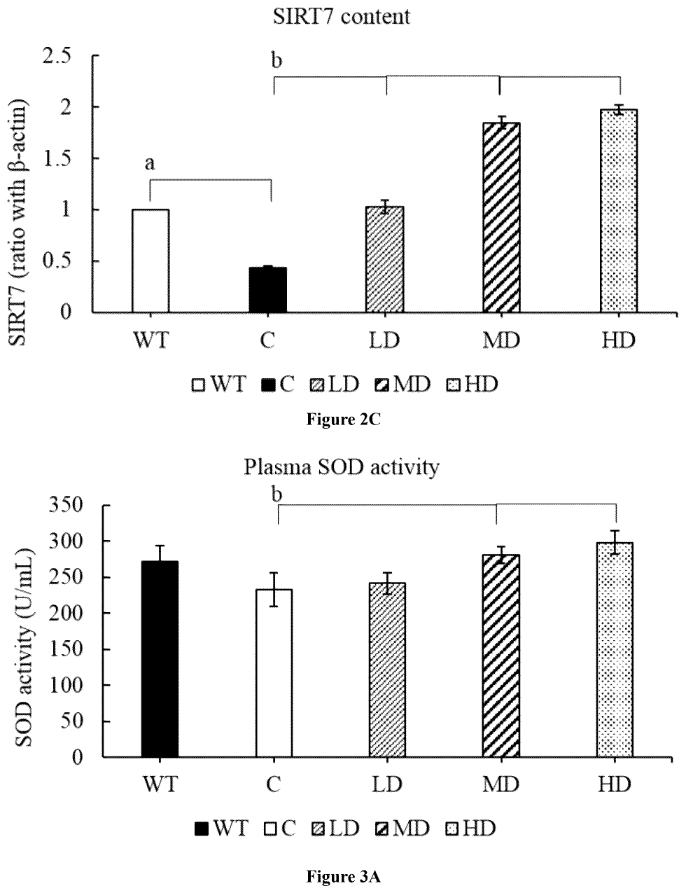

In the experimental results of this invention (FIGS. 2A-2C, 3A-3B, 4A-4C), a indicates a statistically significant difference compared to the WT group (p<0.01), while b indicates a statistically significant difference compared to the C group (p<0.01).

### 3.1. Delayed Aging-Related Gene Expression Test

Aging in mice was induced using D-galactose, followed by administration of three different doses (LD, MD, HD) of the test substance over 12 weeks. After the treatment period, blood samples were collected, and blood cells were isolated to analyze the expression of aging-related genes. Among these genes: SIRT1 regulates homologous chromosome recombination and the repair of DNA breaks. SIRT6 oversees the correction of base-pair mismatches and promotes homologous chromosome recombination. SIRT7 facilitates the ligation of broken DNA ends, contributing to the repair of DNA damage.

As shown in FIGS. 2A-2C, after 12 weeks of continuous administration, groups treated with different concentrations of the fermented composition developed in this study demonstrated a significant increase in the expression levels of SIRT1, SIRT6, and SIRT7. Compared to the aging group, this indicates a marked improvement in DNA repair capacity.

### 3.2. Blood Biochemical Experiments

Aging was induced in mice using D-galactose, followed by oral administration of low, medium, and high doses of the test substance for 12 weeks. Blood samples were collected to analyze the levels of antioxidant markers SOD and GSH. Since aging is considered a consequence of excessive oxidative stress leading to cellular damage, higher levels of these antioxidant markers indicate a stronger capacity to delay aging.

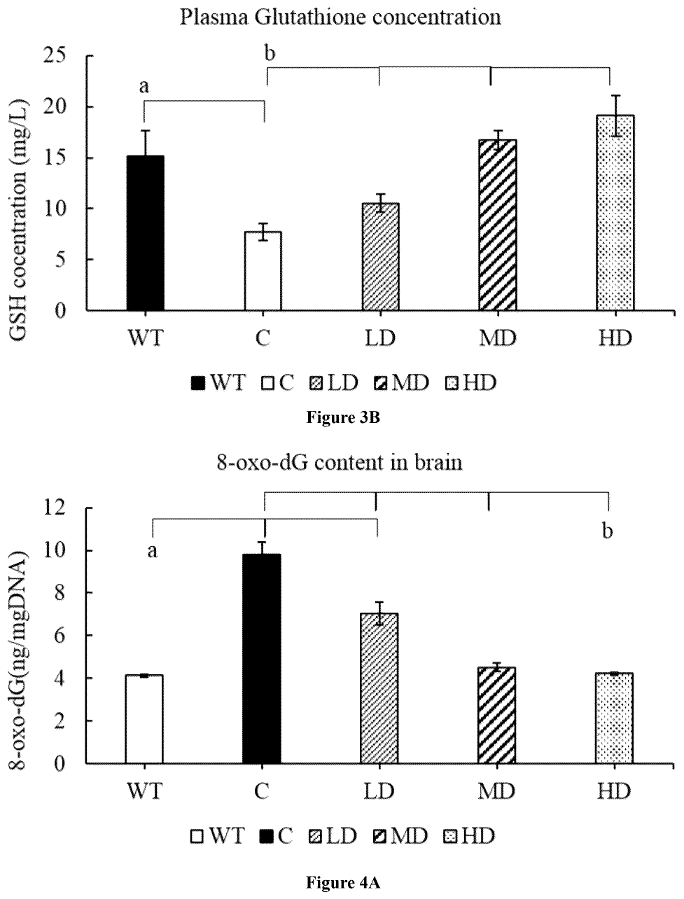

As shown in FIGS. 3A-3B, after 12 weeks of continuous administration, all groups treated with varying concentrations of the fermented composition demonstrated a significant increase in antioxidant substance levels. Compared to the aging group, the treated groups exhibited markedly improved antioxidant capacity.

### 3.3. Determination of Biological Activity Index of Tissue and Organ Aging

Mice were subjected to aging induction using D-galactose and subsequently administered with low, medium, and high doses of the test substance for 12 weeks. Organ tissues, including brain, liver, and heart, were collected as samples for analysis. The concentration of oxidized molecules within these tissues was measured, with 8-oxo-dG serving as a biomarker frequently present in humans. Higher levels of 8-oxo-dG indicate accelerated cellular aging within the tissues and organs.

The brain is the primary organ affected by aging, with oxidative stress leading to the formation of 8-oxo-dG within tissues. Higher concentrations of this oxidative byproduct are indicative of more severe aging phenomena. Reducing oxidative molecule concentrations in various organs can have significant health benefits. For instance, lowering oxidative stress in the brain may reduce the risk of developing Alzheimer's or Parkinson's disease. In the heart, it can decrease the risk of cardiovascular disease, while in the liver, it may alleviate metabolic burden and stabilize liver function indices.

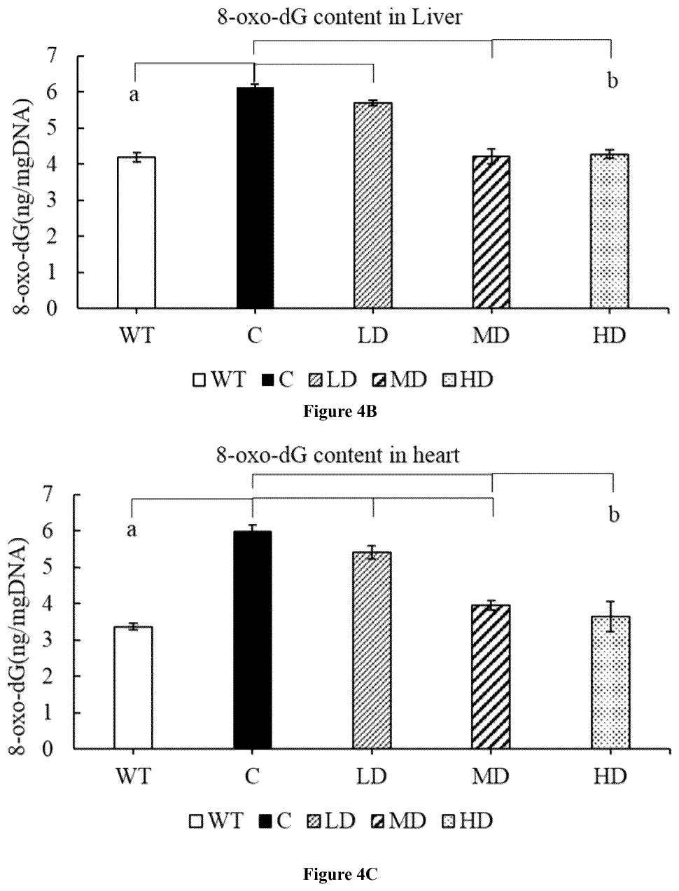

As shown in FIGS. 4A-4C, the levels of aging-related byproducts in the experimental groups were comparable to those of the young mouse group, indicating a delay in brain aging. Similarly, the liver and heart, which are also prone to aging, demonstrated a reduced production of aging-related byproducts, suggesting the ability to retard aging processes in these organs as well.

### 3.4. Survival Period Test

Aging in mice was induced using D-galactose, followed by administration of three different dosages of the test compound until natural death. Mortality data were collected, and the mean lifespan and maximum survival days were analyzed. Statistical analysis was performed to determine significant differences, evaluating whether the invention has a life-extending effect.

In this survival trial, a long-term observation of mice was conducted to directly evaluate the potential life-extending effects of the intervention. Mice were administered three different dosages (HD, MD, LD) daily until natural death. Data were collected and statistically analyzed to assess the outcomes.

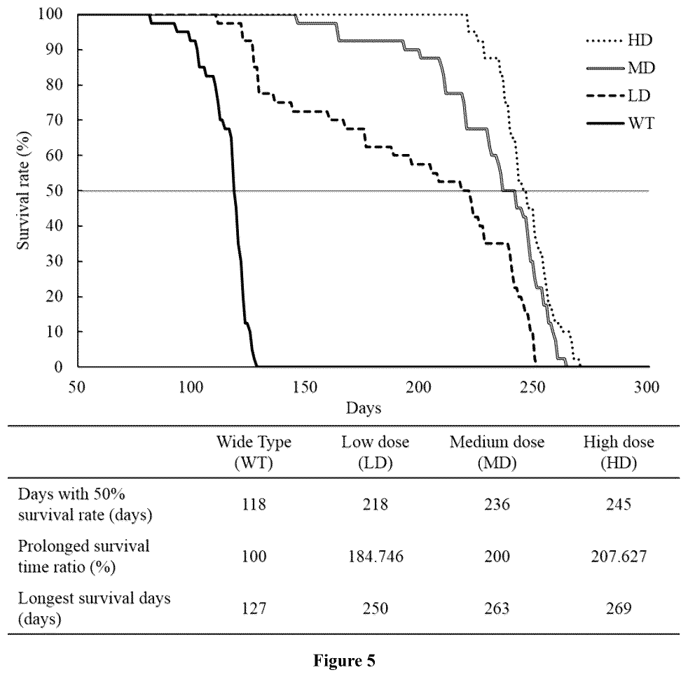

As shown in FIG. 5, when the mortality rate reached 50%, the control group (which did not receive the fermented composition of the present invention) had a survival time of 118 days. However, the group that received the fermented composition at the recommended dosage exhibited a 200% increase in lifespan. The results demonstrate that the fermented composition of the present invention significantly enhances lifespan. Specifically, the control group had a maximum survival of 127 days, while the group receiving the fermented composition at the recommended dosage survived for up to 263 days, extending their lifespan by 207%.

Based on animal studies, the fermented composition of the present invention helps delay aging. It reduces the levels of DNA oxidation products, such as 8-oxo-dG, effectively mitigates lipid peroxidation in the brain, and decreases the extent of oxidative damage in brain tissues. As a result, it helps improve brain oxidative stress and delays brain aging.

The experimental results confirm that the fermented composition of the present invention effectively delays aging by modulating aging-related markers, including the expression of genes associated with aging, enhancing antioxidant indicators such as SOD and GSH levels, and reducing the concentration of oxidative products in tissues and organs.

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims.

1. A method for preparing a fermentation composition having an NMN delayed aging effect, comprising:
(1) a vegetable and fruit extraction step: respectively carrying out squeezing treatment on various vegetables and fruits in a physical manner, pressing vegetables and fruits until the fruits or the fruiting bodies are in a fragment shape, and meanwhile, reserving juice and pomace or fruiting bodies to obtain a plurality of first extracts;
(2) a negative pressure wall breaking method: respectively adding 0.15-1.5% of brown sugar to enhance the isotonicity of the first extract, and simultaneously utilize a negative pressure extraction method in a near-vacuum environment of 35-45 cmHg, continuously extracting for 3-15 days, this process will disrupt the cell walls of fruits and vegetables, releasing intracellular nutrients and polysaccharides to obtain multiple secondary extracts;
(3) a low-temperature fermentation stage: a yeast strain, at a ratio of 0.5-1.5% (10⁷ CFU/mL), is inoculated into the secondary extracts prepared in step (2), the fermentation is carried out at a controlled temperature of 2-12° C. for 3-15 day; during this stage, the yeast remains in a low-activity state, allowing enzymatic activity to dominate over microbial activity, which slows down carbohydrate consumption, this process aids in preservation during fermentation, enhances the decomposition of fibrous polysaccharides, and promotes the breakdown of cell walls, ultimately yielding multiple primary fermentation products;
(4) a deep fermentation stage: a lactic acid bacterium, at a ratio of 0.5-1.5% (10⁷ CFU/mL), along with 0.15-0.25% isomalto-oligosaccharides, is inoculated into the primary fermentation products prepared in step (3); the fermentation is conducted at a controlled temperature of 20-35° C. for 5-25 days, during this stage, the lactic acid bacteria metabolize sugars to produce lactic acid, which lowers the pH to achieve a preservative effect, simultaneously, the increased acidity reduces viscosity, resulting in the production of multiple secondary fermentation products;
(5) a chelation fermentation stage: an acetic acid bacterium, at a ratio of 0.5-1.5% (10⁷ CFU/mL), along with 2.5-5.5% D-sorbitol, is inoculated into the secondary fermentation products prepared in step (4); the fermentation process is carried out at a controlled temperature of 10-25° C. for 5-20 days, during this stage, in an oxygen-rich environment, glucose is consumed to produce acetic acid, ethanol is metabolized, acidity is increased, and viscosity is reduced, resulting in the production of multiple tertiary fermentation products; and
(6) drying and granulating: the tertiary fermentation products prepared in step (5) are individually filtered to obtain multiple fermentation liquids, which are retained, these fermentation liquids are then mixed in equal proportions and subjected to spray drying to form granules, resulting in the fermentation composition.

2. The preparation method according to claim 1, wherein the vegetable and fruit extraction step further comprises mixing the vegetable and fruit with water.

3. The preparation method according to claim 1, wherein the vegetable and fruit are one or more of truffle, mushroom fruiting body, avocado, tomato or edamame.

4. The preparation method according to claim 3, wherein when the vegetables and fruits in the vegetable and fruit extraction step are truffle or mushroom fruiting bodies, before squeezing in a physical manner, the vegetable and fruit extraction step further comprises a step of soaking in hot water at 65-100° C. for 25-35 minutes.

5. The preparation method according to claim 2, wherein the mixing mass ratio of the vegetables and fruits to the water is respectively as follows: the ratio of the truffle or the mushroom fruiting bodies to the water is 1:40; the ratio of the avocado to the water is 1:25; the ratio of the tomato to the water is 1:1; and the ratio of the edamame to the water is 1:30.

6. The preparation method according to claim 1, wherein the secondary extract has a sugar degree of 21-28° BX; the first fermentation product has a sugar degree of 15-34° BX; the second fermentation product has a sugar degree of 15-38° BX; and the tertiary fermentation product has a sugar degree of 10-50° BX.

7. The preparation method according to claim 1, wherein the pH value of the secondary extract is 5.5-7, the pH value of the first fermentation product is 5-6.5, the pH value of the second fermentation product is 4.5-5.5, and the pH value of the tertiary fermentation product is 3-3.5.

8. The preparation method according to claim 1, wherein the lactic acid bacteria are selected from one of Lactobacillus plantarum (L. plantarum), Lactobacillus bulgaricus (L Delbrueckii), Lactococcus lactis (L Lactis), Lactobacillus acidophilus or Bifidum.

9. The preparation method according to claim 1, wherein the saccharomyceses or the acetic acid bacteria are one selected from the group consisting of S fibuligera, S cerevisit, P. faciens, S. pombe, A Hansenii, A Xylimim, or A Surboxy.

10. A fermentation composition with NMN postponed aging effect prepared by the method according to claim 1, comprising a vegetable and fruit raw material, wherein the vegetable and fruit raw material comprises one or more of truffle, mushroom fruiting body, avocado, tomato or edamame, and the fermentation composition obtained by the preparation method.

11. A method for postponing aging, the method comprises administering to the subject a fermentation composition of claim 10, wherein postponing aging is postponing the expression of aging-related genes, improving the content of SOD and GSH of antioxidant indicators, and reducing the oxide concentration of tissues and organs.

12. The method of claim 11, wherein the drug can be used to postpone aging of the heart, brain and liver.
